(19) United States
(12) Patent Application Publication
(10) Pub. No.: US 2006/0027454 A1
(43) Pub. Date: Feb. 9, 2006

(54) Procedure for the fractionation of proteins by using sequential ion exchange and hydrophobic interaction chromatography as prefractionation steps before analysis by two dimensional electrophoresis

(76) Inventor: Augustine DiNovo, Charleston, SC (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/199,601

(22) Filed: Aug. 9, 2005

Related U.S. Application Data
(60) Provisional application No. 60/599,957, filed on Aug. 9, 2004.

(51) Int. Cl. C07K 1/26

(52) U.S. Cl. 204/450; 530/416

(57) ABSTRACT

After the sequencing of the human genome, great interest has developed in trying to discern the complementary proteome of humans and other species. The present disclosure provides devices, systems, and methods for proteomic fractionation that may increase the number of protein spots visualized by 2DE analysis, and may allow enrichment of proteins normally not detectable by standard 2DE analysis. According to some embodiments of the disclosure, devices, systems, and methods of the disclosure relate to fractionating a proteome on the basis of surface charge, hydrophobicity, isoelectric point and/or size.

### RELATED APPLICATION

This application claims priority to U.S. Provisional Patent Application Ser. No. 60/599,957, filed Aug. 9, 2004 and entitled “PROCEDURE FOR THE FRACTIONATION OF PROTEINS BY USING SEQUENTIAL ION EXCHANGE AND HYDROPHOBIC INTERACTION CHROMATOGRAPHY AS PREFRACTIONATION STEPS BEFORE ANALYSIS BY TWO DIMENSIONAL ELECTROPHORESIS” the contents of which are hereby incorporated in their entirety by reference.

### TECHNICAL FIELD OF THE DISCLOSURE

The present disclosure relates to devices, systems, and methods for fractionating proteins.

### BACKGROUND OF THE DISCLOSURE

A number of approaches have been used to try to increase the number of proteins identified by 2DE analysis. Some researchers have prefractionated starting material into organelle specific fractions. This can be either a simple two-part fractionation such as membrane bound and non-membrane bound proteins, or more complicated separations using ultracentrifugation to separate out specific organelles. These methodologies require expensive equipment (an ultracentrifuge), and only enriches proteins of a given organelle rather than truly improving resolution. It may be useful in specifically investigating the expression patterns of a given organelle or compartment, but may be too cumbersome to be applied in looking at all of the proteins in the proteome.

According to another prefractionation methodology, differential solubility may be used to separate proteins into fractions. Raw material may be sequentially processed in buffers with progressively stronger detergents, and chaotropic agents, to separate proteins based upon their hydrophobic/hydrophilic characteristics. Unfortunately, such methods result in a lot of proteins being present in more than one fraction which increases the difficulty of analysis. Other methods use other electrophoretic techniques such as preparative isoelectric focusing or preparative electrophoresis to prefractionate raw material into discrete fractions. These also require special relatively expensive equipment, and may be time consuming. Use of a liquid phase isoelectric focusing apparatus has been the most vigorously pursued of the electrophoretic approaches. Devices in commercial production such as Gradiflow (Gradipore-Frenchs Forest, NSW, Australia) or Rotofor Cell (Biorad) have been applied to this field but require fairly large volumes, and have difficulties with identifying proteins with isoelectric points near their pI cut offs.

### SUMMARY OF THE DISCLOSURE

The present disclosure provides devices, systems, and/or methods for performing dual chromatography as a prefractionation procedure, e.g., for proteomic analysis of global protein expression using two dimensional electrophoresis. Protein fractions produced according to some embodiments of the disclosure may have a low incidence of the same proteins occurring in different fractions. This incidence may be lower than generally observed in other prefractionation approaches that operate on its scale. In some embodiments, methods of the disclosure use a “bind/no bind” load and elution scheme to create fractions, simplifying the chromatography.

The present disclosure provides methods of protein fractionation. Advantageously, the methods comprise prefractionation steps that greatly improve resolution of proteins. The methods of the disclosure may be particularly useful in proteomics applications.

In some embodiments of the disclosure the method comprises prefractionation by anionic exchange chromatography, hydrophobic interaction chromatography, and combinations thereof. A non-limiting example of a proteomic protein fractionation method of the disclosure comprises (a) applying a protein mixture to an anion exchange column, (b) eluting the anion exchange column with a high salt buffer, (c) applying the eluate to a hydrophobic interaction chromatography column, (d) eluting the hydrophobic interaction chromatography column with a low salt buffer, (e)(optional) eluting the hydrophobic interaction chromatography column with a low salt buffer comprising an organic solvent, and (f) fractionating the hydrophobic interaction chromatography column eluate (low salt eluate, low salt plus solvent eluate, and/or a combined low salt/low salt plus solvent eluate) by isoelectric focusing and polyacrylamide electrophoresis.

Embodiments of the present disclosure may provide separation of protein mixtures into a small number of fractions with defined characteristics that show only limited overlap between fractions compared to other methods. Devices, systems, and methods of the disclosure may be partially or completely automated for simple processing. According to some embodiments, low abundance proteins may be concentrated, e.g., basic proteins, and allow them to be visualized in 2DE analysis where they wouldn't be visualized using normal 2DE. See FIG. 2. In some embodiments, a device, system, and/or method of the disclosure may utilize protein surface hydrophobicity as a separation axis to gather physiochemical information. The amount of physiochemical information gathered may be more than obtained by other methods that prefractionate by size or charge only. The methodology may require only basic largely disposable bench top chromatography supplies to perform, and is therefore a low cost means of increasing the sensitivity of 2DE analysis.

### BRIEF DESCRIPTION OF THE DRAWINGS

Some embodiments of the disclosure may be understood by referring, in part, to the following description and the accompanying drawings, wherein:

FIG. 1 shows a basic sequential prefractionation scheme using a three fraction strategy.

FIG. 2A shows a 2DE analysis of HT-29 human carcinoma cell line cytosol fractionated using traditional methods. The iso-electric focusing pH range was 3-10 and the SDS-PAGE dimension utilizes a 12% acrylamide gel.

FIG. 2B shows a 2DE analysis of HT-29 human carcinoma cell line cytosol fractionated using a method comprising an anion exchange chromatography prefractionation step (Fraction 1). The iso-electric focusing pH range was 3-10 and the SDS-PAGE dimension utilizes a 12% acrylamide gel.

FIG. 2C shows a 2DE analysis of HT-29 human carcinoma cell line cytosol fractionated using a method comprising an anion exchange chromatography prefractionation step and a hydrophobic interaction chromatography step (Fraction 2). The iso-electric focusing pH range was 3-10 and the SDS-PAGE dimension utilizes a 12% acrylamide gel.

FIG. 2D shows a 2DE analysis of HT-29 human carcinoma cell line cytosol fractionated using a method comprising an anion exchange chromatography prefractionation step and a hydrophobic interaction chromatography step with elution (Fraction 3). The iso-electric focusing pH range was 3-10 and the SDS-PAGE dimension utilizes a 12% acrylamide gel.

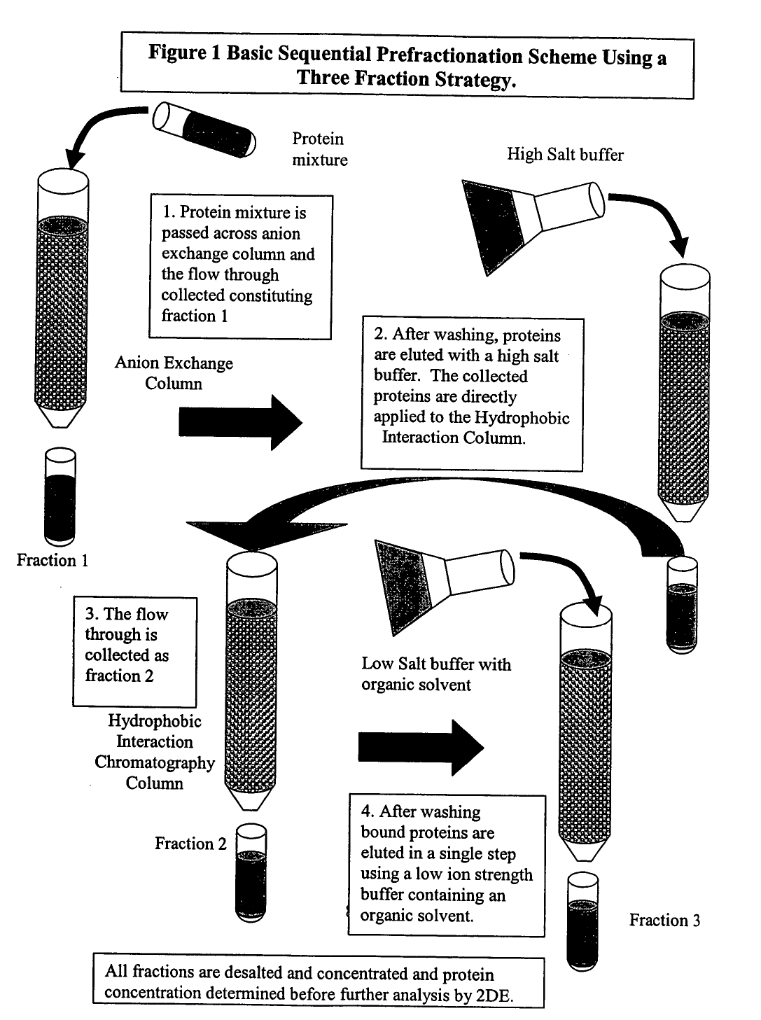

### DETAILED DESCRIPTION OF THE PREFERRED EMBODIMENTS OF THE PRESENT DISCLOSURE

After the sequencing of the human genome, great interest has developed in trying to discern the complementary proteome of humans and other species. At the onset of this type of research the workhorse of parallel analysis of large numbers of proteins was two dimensional electrophoresis (2DE). While powerful, the method suffers from a number of short-comings, and thus research has now focused on means of either alternative superior methods of proteomic analysis or ways of improving 2DE. The means researchers have used to try to improve the 2DE analytical process include changes in the 2DE analysis method itself or by introducing new processing steps before the 2DE procedure is applied. This has been termed prefractionation, and its goal is to produce a small number of discrete fractions that are each independently analyzed by 2DE. The present disclosure provides devices, systems, and methods for proteomic fractionation that may increase the number of protein spots visualized by 2DE analysis, and may allow enrichment of proteins normally not detectable by standard 2DE analysis.

Devices, systems, and methods are provided for separation of a mixture of proteins into several discreet fractions having specific physiochemical properties. A mixture of proteins, according to some embodiments has at least two proteins with differing structures (e.g., primary, secondary, tertiary, or quaternary). Is some embodiments, a mixture of proteins may contain substantially all proteins in the proteome of an organism, a tissue and/or cell type. Fractions may be further analyzed by one or more additional discriminating techniques including, without limitation, two dimensional electrophoresis (2DE) or high resolution chromatography. A protein mixture may be prepared by standard techniques including removal of insoluble materials. Additionally, protein concentration may be determined for the mixture, e.g., when quantitative comparisons of individual proteins between two or more experimental treatments are involved. When there are multiple experimental treatments involved each treatment may be processed separately.

In some embodiments, a protein mixture may be sequentially subjected to two chromatographic techniques. First a known quantity of a protein mixture may be applied to an anion exchange column. This column may be selected to bind all proteins with a significant negative surface charge at the pH of the buffer chosen. Proteins that do not bind to the anion exchange column may be collected as fraction 1 and may be characterized as “high pI fraction”.

Bound proteins may be eluted from the anion exchange column using a high salt buffer (e.g., about 0.5 M or higher). Eluted proteins may be applied (e.g., directly) to second column including, without limitation, a hydrophobic interaction column. According to some embodiments of the disclosure, a hydrophobic interaction column may utilize a C4 (t-butyl) functional group immobilized on a hydrophobic support (as opposed C4 functional moiety attached to a silica or related supports typically used in reversed phase chromatography).

Hydrophobic interaction chromatography may be conducted in a high salt buffer (e.g., about 0.5 M or higher) producing an environment where moderately to weakly hydrophobic proteins will bind to the chromatography resin. (In reversed phase chromatography, only proteins with moderate to high hydrophobicity will bind.) Again proteins that do not interact with the column are collected and this fraction is defined as low pI hydrophilic fraction (fraction 2).

One of ordinary skill in the art will recognize that the exact concentration of salt solution used to elute an ion exchange column or a hydrophobic interaction column will influence the nature of the proteins eluted. Thus, in some embodiments of the disclosure, a loading and/or elution buffer may have a high salt concentration. For example, a high salt buffer may have a salt concentration of about or over 500 mM, about or over 1.0 M, and/or about or over 1.7 M. In other embodiments, a loading or elution buffer may have a low salt concentration. For example, a low salt buffer may have a salt concentration of about or under 500 mM, about or under 100 mM, about or under 50 mM, and/or about or under 25 mM. In some embodiments, a salt gradient may be used to elute a column.

In some embodiments, bound proteins may be eluted from this column either with two sequential elution washes with first a low salt buffer (e.g., about or less than 0.1 M), followed by the same low salt buffer also containing an organic solvent such as methanol (e.g., 30% v/v methanol). In other embodiments, bound proteins may be eluted in a single wash using a low salt buffer containing an organic solvent. (This elution scheme is actually a combination of hydrophobic interaction chromatography which typically uses low salt for elution and reversed phase chromatography which typically uses an organic solvent for elution.) If a single fraction elution scheme is used the fraction is considered the low pI, hydrophobic fraction. If a two step elution scheme is used the first elution fractions is considered the mildly hydrophobic low pI fraction, and the second fractions is considered the strongly hydrophobic low pI fraction.

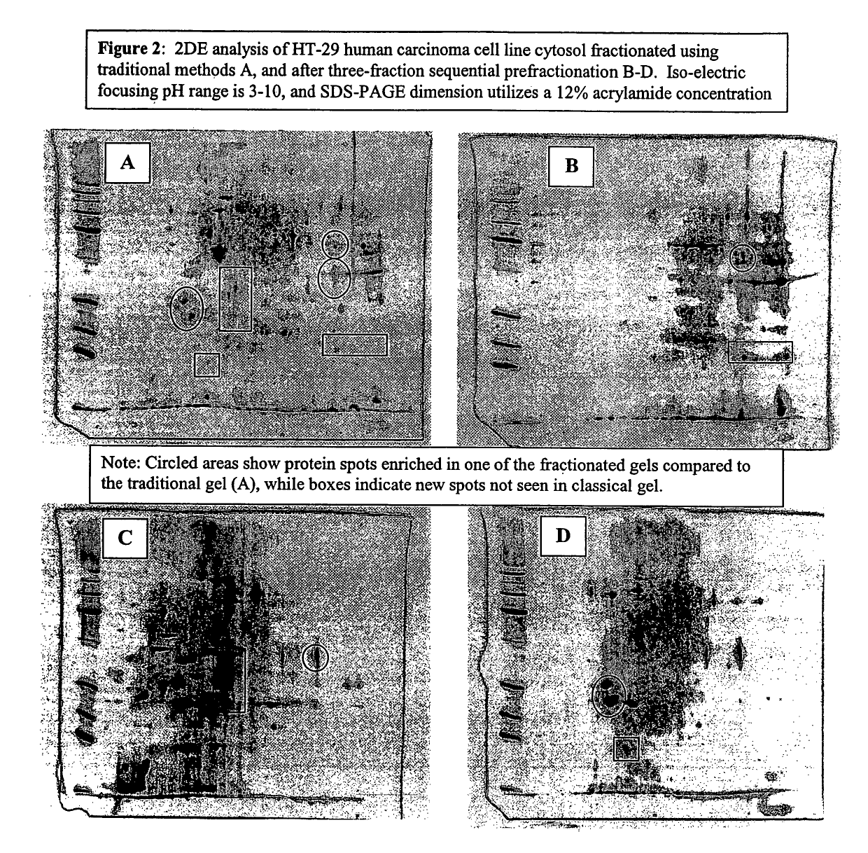

Typically, each of the fractions may be concentrated using standard practices. In some embodiments, all fractions except fraction 1 may be desalted, again by standard practices, to remove the high salt and organic solvents which may interfere with later high resolution analysis. Total protein may be determined for each fraction. For two dimensional electrophoresis, an appropriate quantity of each fraction is diluted in standard denaturing buffer and loaded onto IPG isoelectric focusing strips for analysis. For high resolution chromatography, an appropriate quantity is loaded into a sample loop without modification. FIG. 2 shows 2DE analysis of the HT-29 human colon carcinoma cell line with standard 2DE and with the 3 fraction-prefractionation scheme described above.

A device and/or system of the disclosure may include a first, second and third protein fractionator. A first protein fractionator may include an ion exchange column and one or more connectors. A second protein fractionator may include a hydrophobic interaction column and one or more connectors. A third protein fractionator may include a size-fractionation matrix, an isoelectric focusing matrix, and combinations thereof. A third protein fractionator may further include one or more connectors. Connectors may link one fractionator to another such that eluate from one fractionator is intermittently, continuously, or conditionally fed into another fractionator. For example, a device may include an anion exchange column connected to a hydrophobic interaction column, which is in turn connected to a 2DE gel and the connections allow passage of eluate only when a desired solvent and/or salt concentration (or range) is present in the eluate. A system of the disclosure may include one or more dispensers that meter the size of fractions collected from a fractionator and/or the amount, kind, and/or concentration of buffer applied to a fractionator. A system of the disclosure may include a power source, potentiostat (e.g., to control current to the 2DE gel), a processing device (e.g., a processor), and/or a display. Systems and devices of the disclosure may be configured to handle sample volumes of more than one milliliter or may be miniaturized to handle smaller sample volumes (e.g., microliters, nanoliters, or less).

### EXAMPLE 1

Four T-75 flasks of HT-29 carcinoma cell cultures were grown until confluent in McCoy's modified medium (Gibco) with 10% FBS, and 1% Penicillin-Streptomycin (Gibco). Media was removed and cultures were rinsed two times with PBS, then lysed with a low ionic strength lysis buffer ((50 mM Tris, pH 7.5, 10 mM DTT, and a protease Inhibitor cocktail (Sigma diluted 100:1), followed by 3 rapid freeze/thaw cycles. Individual lyses were pooled and centrifuged for 20 minutes at 24,000×G at four degrees Celsius, followed by filtering with a 0.2 μm syringe filter. Total protein was determined by Bradford's dye binding assay. An aliquot was diluted 5:1 in 2DE lysis buffer (7 M urea, 2 M thiourea, 4% CHAPS, 4 mM tributyl phosphine (TBP), 0.5% ampholyte solution, and a trace of bromophenol blue) and analyzed by 2DE and is designated the traditional method.

Three milligrams of the cytosolic extract was applied to an ion exchange column (Poly-Prep gravity column (Biorad) containing 1 ml bed volume of MacroPrep Q ion exchange media (Biorad)) pre-equilibrated with 5 bed volumes of 50 mM Tris, pH 7.5. The flow-through was collected as fraction 1. After a 4 bed volume wash with 50 mM Tris, pH 7.5, the bound protein was eluted with 1.7 M ammonium sulfate in 25 mM Tris pH 7.5.

The elutate was then directly applied to a hydrophobic interaction chromatography column (Poly-prep column (Biorad) containing 1 ml bed volume of Macro-Prep HIC media (Biorad)) pre-equilibrated with 5 volumes of 1.7 M ammonium sulfate in 25 mM Tris pH 7.5. The flow-through was collected as fraction 2. After a 4 volume wash with 1.7 M ammonium sulfate in 25 mM Tris pH 7.5, the bound protein was eluted with 25 mM Tris, pH 7.5 containing 30% methanol, as fraction 3. All three fractions were concentrated using Ultra 4 centrifugal concentrators (Millipore), and fractions 2 and 3 were also desalted using the same concentrators but re-diluting the concentrate with 25 mM Tris, pH 7.5 twice. Total protein was determined for all fractions.

Each fraction was diluted 5:1 in 2DE lysis solution. For 2DE, 40 μg of protein was loaded overnight onto 11 cm immobilized pH gradient (IPG) strips (pH range 3-10) by the passive rehydration method. Iso-electric focusing (IEF) was conducted using a Multiphor II (GE Healthcare). After IEF, IPG strips were equilibrated for 20 minute in an equilibrium buffer (6 M urea, 50 mM Tris pH 6.8, 2% w/v SDS, 30% glycerol, and a trace of bromophenol blue). The second dimension (SDS PAGE) was conducted on a Hoeffer 600 (GE Healthcare) vertical electrophoresis unit using 12% total acrylamide homogeneous gels. After electrophoresis, gels were silver stained by the method of Blum. Stained gels were scanned on an HP psc 750 printer/scanner/copier. Results are shown in FIGS. 2A-D.

As will be understood by those skilled in the art, other equivalent or alternative devices, systems, and/or methods for fractionating proteomic proteins and/or polypeptides can be envisioned without departing from the essential characteristics thereof. For example, devices of the disclosure may be manufactured in either a handheld or a tabletop configuration, and may be operated intermittently or continuously. Moreover, individuals skilled in the art would recognize that additional chromatographic techniques or separation methods may be incorporated, e.g., to partially or completely remove lipids, carbohydrates, and/or nucleic acids. Also, the temperature, pressure, and acceleration (e.g., spin columns) at which each step is performed may be varied. Additionally, detectors may be configured and positioned to detect the conditions, progress, and/or results of fractionation. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

What is claimed is:

1. A method of proteomic protein fractionation comprising:
applying a protein mixture to an anion exchange column,
eluting the anion exchange column with a high salt buffer to form an anion exchange eluate,
applying the anion exchange eluate to a hydrophobic interaction chromatography column,
eluting the hydrophobic interaction chromatography column with a low salt buffer to form a low salt eluate, and
fractionating the low salt eluate by isoelectric focusing and polyacrylamide electrophoresis.

2. A method according to claim 1 further comprising eluting the hydrophobic interaction chromatography column with a low salt buffer comprising an organic solvent to form a low salt plus solvent eluate, and fractionating the low salt plus solvent eluate by isoelectric focusing and polyacrylamide electrophoresis.

3. A method according to claim 1, wherein the hydrophobic interaction chromatography column comprises a C4 (t-butyl) functional group immobilized on a hydrophobic support.

4. A method according to claim 1, wherein the isoelectric focusing pH range is from about 3 to about 10.

5. A method according to claim 1, wherein the polyacrylamide electrophoresis is performed on a gel comprising from about 10% to about 12% by weight polyacrylamide.

6. A method according to claim 1, wherein the high salt buffer comprises ≧about 0.5 M salt.

7. A method according to claim 1, wherein the low salt buffer comprises ≦about 0.1 M salt.

8. A method according to claim 1, wherein the low salt buffer comprising an organic solvent comprises ≦about 0.1 M salt.

9. A method according to claim 1, wherein the protein mixture comprises from two to about 100,000 proteins.

10. A method according to claim 1, wherein the protein mixture comprises substantially every protein in an organism's proteome.

11. A method according to claim 10, wherein the organism is selected from the group consisting of microbes, invertebrates, and vertebrates.

12. A method according to claim 11, wherein the vertebrate is selected from the group consisting of humans and non-human mammals.

13. A device for proteomic protein fractionation comprising:
a first fractionator configured to receive a protein mixture,
a second fractionator in fluid contact with the first fractionator, and
a third fractionator in fluid contact with the first or second fractionator,
wherein the first fractionator comprises an anion exchange column, the second fractionator comprises a hydrophobic interaction chromatography column, and the third protein fractionator comprises a size-fractionation matrix and an isoelectric focusing matrix.

14. A device according to claim 13, wherein the size-fractionation matrix comprises polyacrylamide.

15. A device according to claim 13, wherein the device is configured to receive the protein mixture in a solution volume ≧about 1.0 mL.

16. A device according to claim 13, wherein the device is configured to receive the protein mixture in a solution volume ≦about 1.0 mL.

17. A system for proteomic protein fractionation comprising:
a first fractionator configured to receive a protein mixture,
a first fractionator eluate connector in fluid contact with the first fractionator and configured to collect eluate from the first fractionator;
a second fractionator in fluid contact with the first fractionator eluate connector;
a second fractionator eluate connector in fluid contact with the second fractionator and configured to collect eluate from the second fractionator; and
a third fractionator in fluid contact with the second fractionator eluate connector,
wherein the first fractionator comprises an anion exchange column, the second fractionator comprises a hydrophobic interaction chromatography column, and the third protein fractionator comprises a size-fractionation matrix and an isoelectric focusing matrix.

18. A device according to claim 17, wherein the first fractionator eluate connector is configured to meter the flow leaving the first fractionator into the second fractionator.

19. A device according to claim 18 further comprising a processor operably linked to the first fractionator eluate connector, wherein the processor conditionally regulates the metered flow.

20. A device according to claim 19, wherein the conditional regulation of eluate flow is conditioned upon salt concentration of the eluate.

21. A device according to claim 17, wherein the second fractionator eluate connector is configured to meter the flow leaving the second fractionator into the third fractionator.

22. A device according to claim 21 further comprising a processor operably linked to the second fractionator eluate connector, wherein the processor conditionally regulates the metered flow.

23. A device according to claim 22, wherein the conditional regulation of eluate flow is conditioned upon salt concentration of the eluate.
